(19) United States
(12) Plant Patent Application Publication
(10) Pub. No.: US 2017/0071098 P1
(43) Pub. Date: Mar. 9, 2017

(54) New Guinea impatiens plant named 'DUESSPLAV'

(50) ;

(71) Applicant: Ruth Kobayashi, Carlsbad, CA (US)

(72) Inventor: Ruth Kobayashi, Carlsbad, CA (US)

(73) Assignee: DUMMEN GROUP B.V., De LIER (NL)

(21) Appl. No.: 14/756,461

(22) Filed: Sep. 8, 2015

(51) Int. Cl. A01H 5/00

(52) U.S. Cl. PLT/318.6

(57) ABSTRACT

A new and distinct cultivar of Impatiens plant named ‘Duessplav’ characterized by its upright, outwardly spreading and mounding plant habit; vigorous growth habit; freely branching habit; dark green-colored leaves; early and freely flowering habit; light violet-colored flowers with red purple-colored central stripes and petal bases; and good garden performance.

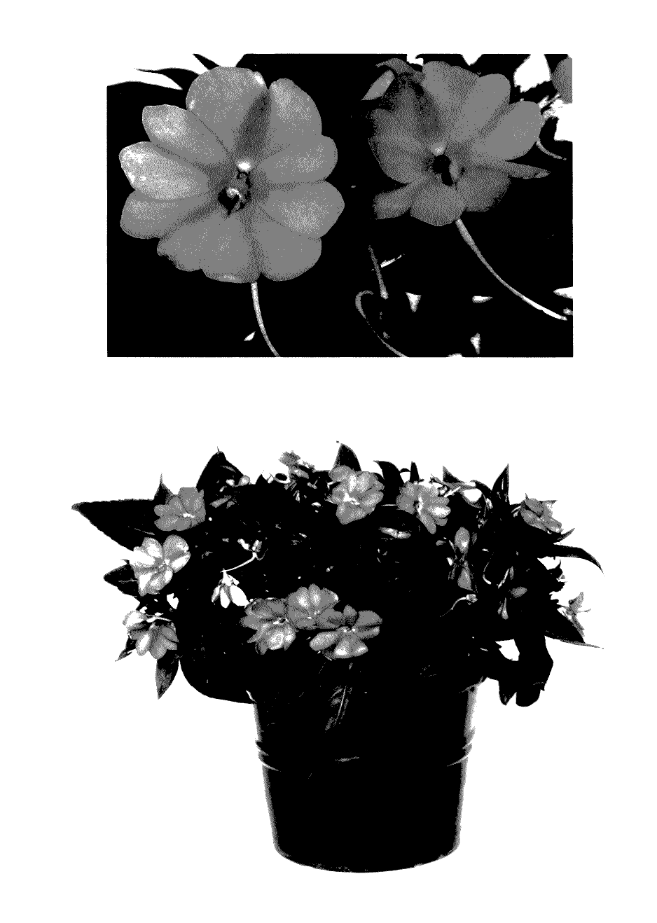

Botanical designation: Impatiens×hybrida.

Cultivar denomination: ‘DUESSPLAV’.

### CROSS-REFERENCED TO CLOSELY RELATED APPLICATIONS:

Title: New Guinea Impatiens Plant Named ‘Duessphotpk’

Filed: Sep. 8, 2015; concurrently with this application

### BACKGROUND OF THE INVENTION

The present invention relates to a new and distinct cultivar of New Guinea Impatiens plant, botanically known as Impatiens×hybrida and hereinafter referred to by the name ‘Duessplav’.

The new Impatiens plant is a product of a planned breeding program conducted by the Inventor in Encinitas, Calif. The objective of the breeding program is to create new uniform Impatiens plants with numerous attractive flowers and good garden performance.

The new Impatiens plant is a naturally-occurring whole plant mutation of a proprietary selection of Impatiens×hybrida identified as code number NN-1166-X0000, not patented. The new Impatiens plant was discovered and selected by the Inventor as a single flowering plant from within a population of plants of the parent selection in a controlled greenhouse environment in Encinitas, Calif. on Dec. 8, 2014.

Asexual reproduction of the new Impatiens plant by terminal vegetative cuttings in a controlled greenhouse environment in Encinitas, Calif. since Jan. 9, 2015 has shown that the unique features of this new Impatiens plant are stable and reproduced true to type in successive generations.

### SUMMARY OF THE INVENTION

Plants of the new Impatiens have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘Duessplav’. These characteristics in combination distinguish ‘Duessplav’ as a new and distinct Impatiens plant:

1. Upright, outwardly spreading and mounding plant habit.

2. Vigorous growth habit.

3. Freely branching habit.

4. Dark green-colored leaves.

5. Early and freely flowering habit.

6. Light violet-colored flowers with red purple-colored petal central stripes and bases.

7. Good garden performance.

Plants of the new Impatiens can be compared to plants of the parent selection. Plants of the new Impatiens differ primarily from plants of the parent selection in flower color as plants of the parent selection have dark lavender pink-colored flowers.

Plants of the new Impatiens can be compared to plants of Impatiens×hybrida ‘Duessphotpk’, disclosed in a U.S. Plant Patent application filed concurrently. Plants of the new Impatiens differ primarily from plants of ‘Duessphotpk’ in flower color as plants of ‘Duessphotpk’ have hot pink-colored flowers.

Plants of the new Impatiens can be compared to plants of Impatiens×hybrida ‘Sakimp006’, disclosed in U.S. Plant Pat. No. 19,501. In side-by-side comparisons conducted in Encinitas, Calif., plants of the new Impatiens differed primarily from plants of ‘Sakimp006’ in the following characteristics:

1. Plants of the new Impatiens were more compact than plants of ‘Sakimp006’.

2. Plants of the new Impatiens and ‘Sakimp006’ differed in flower color as plants of ‘Sakimp006’ have lavender-colored flowers.

Plants of the new Impatiens can also be compared to plants of Impatiens×hybrida ‘SAKIMP017’, disclosed in U.S. Plant Pat. No. 21,753. In side-by-side comparisons conducted in Encinitas, Calif., plants of the new Impatiens differed primarily from plants of ‘SAKIMP017’ in the following characteristics:

1. Plants of the new Impatiens were not as compact as plants of ‘SAKIMP017’.

2. Plants of the new Impatiens and ‘SAKIMP017’ differed in flower color as plants of ‘SAKIMP017’ have deep rose-colored flowers.

### BRIEF DESCRIPTION OF THE PHOTOGRAPHS

The accompanying colored photographs illustrate the overall appearance of the new Impatiens plant showing the colors as true as it is reasonably possible to obtain in colored reproductions of this type. Colors in the photographs may differ slightly from the color values cited in the detailed botanical description which accurately describe the colors of the new Impatiens plant. The photograph at the bottom of the sheet is a side perspective view of a typical flowering plant of ‘Duessplav’ grown in a container. The photograph at the top of the sheet is a close-up view of a typical flowering plant of ‘Duessplav’.

### DETAILED BOTANICAL DESCRIPTION

The aforementioned photographs and following observations, measurements and values describe plants grown during the summer in 15.25-cm containers in a polyethylene-covered greenhouse in Encinitas, California and under cultural practices typical of commercial New Guinea Impatiens production. During the production of the plants, day temperatures ranged from 24° C. to 27° C., night temperatures averaged 18° C. and light levels averaged 4,000 foot-candles. Plants were ten weeks old when the photographs and description were taken. In the following description, color references are made to The Royal Horticultural Society Colour Chart, 2007 Edition, except where general terms of ordinary dictionary significance are used.
Botanical classification: Impatiens×hybrida ‘Duessplav’.
Parentage: Naturally-occurring whole plant mutation of a proprietary selection of Impatiens×hybrida identified as code number NN-1166-X0000, not patented.
Propagation:
   Type.—By terminal cuttings.
   Time to initiate roots, summer and winter.—About five to seven days at day temperatures about 27° C. and night temperatures about 20° C.
   Time to produce a rooted young plant, summer and winter.—About three weeks at day temperatures about 27° C. and night temperatures about 20° C.
   Root description.—Fine, fibrous; white in color, actual color of the roots is dependent on substrate composition, water quality, fertilizer type and formulation, substrate temperature and physiological age of roots.
   Rooting habit.—Freely branching; dense.
Plant description:
   Plant and growth habit.—Upright to outwardly spreading and mounding plant habit; freely branching habit with about nine to eleven lateral branches developing per plant; vigorous growth habit.
   Plant height.—About 18 cm.
   Plant diameter.—About 38 cm.
Lateral branch description:
   Length.—About 17 cm.
   Diameter.—About 1.2 cm.
   Internode length.—About 4 cm.
   Strength.—Strong.
   Aspect.—Initially upright to outwardly spreading.
   Texture.—Smooth, glabrous.
   Color.—Close to 187A.
Leaf description:
   Arrangement.—Opposite or in whorls; simple.
   Length.—About 11 cm.
   Width.—About 5 cm.
   Shape.—Elliptical.
   Apex.—Acuminate.
   Base.—Attenuate.
   Margin.—Serrulate with ciliation.
   Texture, upper and lower surfaces.—Smooth, glabrous.
   Luster, upper surface.—Somewhat glossy.
   Luster, lower surface.—Matte.
   Venation pattern.—Pinnate; arcuate.
   Color.—Developing leaves, upper surface: Close to 146A. Developing leaves, lower surface: Close to 183B. Fully opened leaves, upper surface: Close to 139A; venation, close to 184A. Fully opened leaves, lower surface: Close to 148A tinted with close to 183C; venation, close to 185A.
   Petiole length.—About 1.8 cm.
   Petiole diameter.—About 4 mm.
   Petiole texture, upper and lower surfaces.—Smooth, glabrous.
   Petiole color, upper and lower surfaces.—Close to 184A.
Flower description:
   Flower type and flowering habit.—Single rounded and flat axillary flowers; freely flowering habit, typically about 45 flower buds and open flowers per plant at one time; flowers positioned above and beyond the foliar plane, flowers typically face mostly upright to outwardly.
   Flower longevity.—Flowers typically last about one to two days on the plant under greenhouse conditions; petals self-cleaning, gynoecium persistent.
   Fragrance.—None detected.
   Natural flowering season.—Year-round under greenhouse conditions; in the garden, flowering from spring until fall in temperate regions; early flowering habit, plants typically begin flowering about eight weeks after planting.
   Flower buds.—Length: About 1.8 cm. Diameter: About 1.3 cm. Shape: Ovoid, pointed. Color: Close to N74B and N74C.
   Flower diameter.—About 4.5 cm to 4.7 cm.
   Flower depth.—About 2.2 cm.
   Petals.—Quantity and arrangement: Five per flower in a single whorl; one upper banner petal, two lateral petals and two lower petals. Length, banner petal: About 2 cm. Width, banner petal: About 2.9 cm. Length, lateral petals: About 2.5 cm. Width, lateral petals: About 2 cm. Length, lower petals: About 2.8 cm. Width, lower petals: About 2.4 cm. Shape: Cordate. Apex: Cordate; emarginate. Base: Attenuate. Margin: Entire. Texture, upper and lower surfaces: Smooth, glabrous; velvety. Color: When opening, upper surface: Close to N78A to N78B. When opening, lower surface: Close to N78C to N78D. Fully opened, upper surface: Close to N78A; central stripes (especially on banner petals) and towards the base, close to N74B; main color becoming closer to N78B with development. Fully opened, lower surface: Close to N74C; central stripes, close to N74A; color does not change with development.
   Sepals.—Quantity and arrangement: Three in a single whorl; one modified into an elongated spur. Length: About 2 cm. Width: About 5 mm. Shape: Narrowly deltoid. Apex: Apiculate. Base: Attenuate. Margin: Entire. Texture, upper and lower surfaces: Smooth, glabrous. Color, upper and lower surfaces: Close to 145C to 145D. Spur length: About 4.7 cm. Spur diameter: At the flower, about 3 mm; towards the apex, about 1 mm. Spur texture: Smooth, glabrous. Spur color: Close to 145C to 145D.
   Peduncles.—Length: About 4 cm. Diameter: About 2 mm to 3 mm. Angle: About 45° from stem axis. Strength: Strong; flexible. Texture: Smooth, glabrous. Color: Close to 146C.
   Reproductive organs.—Stamens: Quantity: Five fused at anthers; filaments free. Filament length: Less than 1 mm. Filament color: Close to 1D. Anther size: About 4 mm by 6 mm. Anther shape: Oblong. Anther color: Close to 71D. Pollen amount: Moderate. Pollen color: Close to 158B. Pistils: Quantity per flower: One. Pistil length: About 6 mm. Stigma shape: Rounded. Stigma color: Close to 160C. Style color: Close to 146A. Ovary color: Close to 146A.
   Seeds and fruits.—Seed and fruit production has not been observed on plants of the new Impatiens.
Disease & pest resistance: Plants of the new Impatiens have not been observed to be resistant to pathogens and pests common to Impatiens plants.
Garden performance: Plants of the new Impatiens have been observed to have good garden performance and tolerate temperatures ranging from about 5° C. to about 40° C.

It is claimed:

1. A new and distinct Impatiens plant named ‘Duessplav’ as illustrated and described.
